(19) United States
(12) Patent Application Publication
(10) Pub. No.: US 2002/0094564 A1
(43) Pub. Date: Jul. 18, 2002

(54) Method for extracting and separating metals

(76) Inventors: Jillian F. Banfield, Berkeley, CA (US); Susan A. Welch, Madison, WI (US); Gregory K. Druschel, Madison, WI (US); Matthias Labrenz, Madison, WI (US); John W. Moreau, Oakland, CA (US)

Correspondence Address: Nicholas J. Seay, Quarles & Brady LLP, 1 South Pinckney Street, P O Box 2113, Madison, WI, 53701-2113, US

(21) Appl. No.: 09/907,530

(22) Filed: Jul. 17, 2001

Related U.S. Application Data
(60) Provisional application No. 60/218,716, filed on Jul. 17, 2000.

(51) Int. Cl. C22B 3/18; C12M 1/00

(52) U.S. Cl. 435/262; 423/37; 423/101; 435/289.1

(57) ABSTRACT

A technique is presented for the sequential extraction of nearly pure metal sulfides from solutions containing a mixture of metals, such as acid mine drainage. The technique is based on analysis of naturally occurring biofilms that selectively concentrate zinc, in zinc-sulfide, from a complex natural groundwater solution associated with a subsurface metal-sulfide mine. It was predicted and shown experimentally that release of sulfide ions, due to the activity of sulfate reducing bacteria, leads to sequential precipitation of pure metal sulfide phases from a solution of mixed metal ions, so long as the rate of production of sulfide ions does not exceed the rate of supply of the metal ions. This observation makes possible the design of biochemical processes to harness sulfate-reducing bacteria to separate and recover metals from mixed-metal waste streams.

### CROSS-REFERENCE TO RELATED APPLICATION

This application claims priority from U.S. provisional patent application No. 60/218,716, filed Jul. 17, 2000.

### BACKGROUND OF THE INVENTION

This invention pertains generally to the field of extracting and separating metal ions from complex aqueous solutions containing mixed metals. Particularly suitable uses for this technology are recovering metals from acid mine drainage, a serious environmental contaminant, and from solutions produced by bioleaching.

In active or abandoned mines and mine tailings water seepage can leach both acids and heavy metals to form a solution that is both acidic and a carrier of metal ions. Acid mine drainage is currently a serious source of environmental contamination. These acid metal solutions are often toxic to many life forms, including humans and many, if not all, higher animals. Control of such leachates is often a major objective in the effort to provide environmental remediation for mine sites.

It is known that some microorganisms can exist in the conditions found in acid mine drainage. Such microorganisms can gain energy from the environment by catalyzing a change in the oxidation state of inorganic ions. Microorganisms are also found in environments impacted by acid mine drainage. A subset of these can reduce sulfate to sulfide. Sulfate-reducing bacteria catalyze the kinetically inhibited reaction between organic compounds and aqueous sulfate (SO₄²⁻) to produce sulfide (H₂S). Sulfide ions then can react with dissolved metals to produce insoluble metal sulfides. While the existence of biologically produced metal sulfide deposits in the environment has previously been noted, the microbiological, geochemical and mineralogical conditions giving rise to such deposits can be difficult to decipher completely. The geochemical conditions giving rise to such deposits of single metal sulfide phases have not been elucidated. Some attempts were made to reproduce this phenomenon in the laboratory using bacteria or diffusion-limiting gels. Although these previous studies produced results that can be rationalized by our geochemical model, these authors did not provide a basis for industrial use of the phenomenon (Temple and Le Roux, Econ. Geol. 59:647-655, 1964; Bubela and McDonald, Nature, 221:465-466, 1969; Lambert and Bubela, Mineral. Deposita. 5:97-102, 1970).

### BRIEF SUMMARY OF THE INVENTION

The present invention is summarized in a method for extracting and segregating metals from an aqueous solution containing mixed metal ions, the method including the steps of exposing the solution to a slowly increasing concentration of sulfide ions to selectively precipitate metal sulfides from the solution, and recovering the metal sulfides as they precipitate.

The present invention is also summarized in reactors designed to perform this method.

It is an object of the present invention to make possible the energy efficient and low-temperature extraction and segregation of metals from a mixed metal ion waste stream using a biological organism to assist in the recovery.

It is an advantage of the present invention in that it enables recovery of potential resources from waste streams that would otherwise be environmental contaminants.

Further objects, features and advantages of the invention will be apparent from the following detailed description when taken in conjunction with the accompanying drawings.

### BRIEF DESCRIPTION OF THE SEVERAL VIEWS OF THE DRAWINGS

FIG. 1 is a graph intended to illustrate a part of the science behind this invention. The graph shows increasingly reducing conditions, or increasing concentration of sulfide ions, plotted against the number of moles of metal sulfides precipitated.

FIG. 2 is a graph illustrating the concentrations of metal ions in a solution being processed in accordance with the present invention.

### DETAILED DESCRIPTION OF THE INVENTION

The path to the present invention began with the discovery of aggregates of very small metal sulfide particles in biofilms recovered from an abandoned mine site. The biofilm contained essentially only one metal sulfide. Geochemical modeling was conducted in order to explain the precipitation of a single metal sulfide compound from a complex natural solution. The modeling predicts the production of a series of discrete metal sulfide precipitation events from an aqueous solution of mixed metal ions as sulfide concentration increases over time. Only a single compound precipitates at one time so long as the rate of sulfide production does not exceed the rate of supply of metal ions or the sulfide precipitation kinetics. The investigators here have discovered that, under these conditions, precipitation of each metal sulfide phase buffers the sulfide concentration at a specific value until the supply of the relevant metal is exhausted. This is because metal ions bind sulfide molecules as they are produced, limiting the accumulation of sulfide in solution. Thus the metal ions are sequestered into sulfide phases in order of increasing solubility. This observation makes it possible to design and specify strategies to selectively remove and separate metal ions from mixed metal solutions by sequentially precipitating the metals as metal sulfides. The novel feature of the technology described here is control of the growth of sulfide-reducing bacteria to obtain the step-wise (rather than simultaneous) extraction of individual metal-sulfides from solutions containing multiple metal ions.

The approach described here is based on the low, but variable solubility of metal sulfide minerals. Because metals such as Cu, Cd, Pb, Zn, and Fe display different relative affinities for aqueous sulfide, a specific mineral precipitation sequence is anticipated as aqueous sulfide is produced from sulfate in a system. However, the model predications are only valid so long as the rate of sulfide mineral precipitation is faster than the rate of production of aqueous sulfide. Given the extremely fast rate of metal sulfide precipitation, this condition is relatively easily attainable by matching the supply of metal ions to the sulfide generation rate and by controlling the rate of growth of sulfate-reducing bacteria, which are used to produce the sulfide. The growth of such bacteria can be easily controlled by techniques routinely used by microbiologists. Control of the growth rate, temperature, flow rate, solution chemistry, or other factors permits the system to be used to separate and recover metals, in the form of metal sulfides, in a continuous and efficient manner from a mixed metal solution, such as an acid mine drainage. The method outlined here could be employed to extract specific metals from acid mine drainage solutions, industrial solutions, and waste streams.

The concept of this invention is that a mixed metal solution is introduced into a system in which a sulfate-reducing bacterial culture is grown. Growth of the bacteria results in increasing amounts of sulfide ions. As the concentration of sulfide ions reaches the point of insolubility of a given metal sulfide species, the metal ions of that species combine with the sulfide ions, and that metal sulfide then precipitates from solution. Since the precipitation removes sulfide ions from the solution, the overall concentration of sulfide ions is, in effect, buffered during the precipitation of a metal sulfide species. When all of the ions of the precipitating metal are depleted from the solution, the concentration of sulfide ions begins to rise again until the point of insolubility of the next metal sulfide is reached.

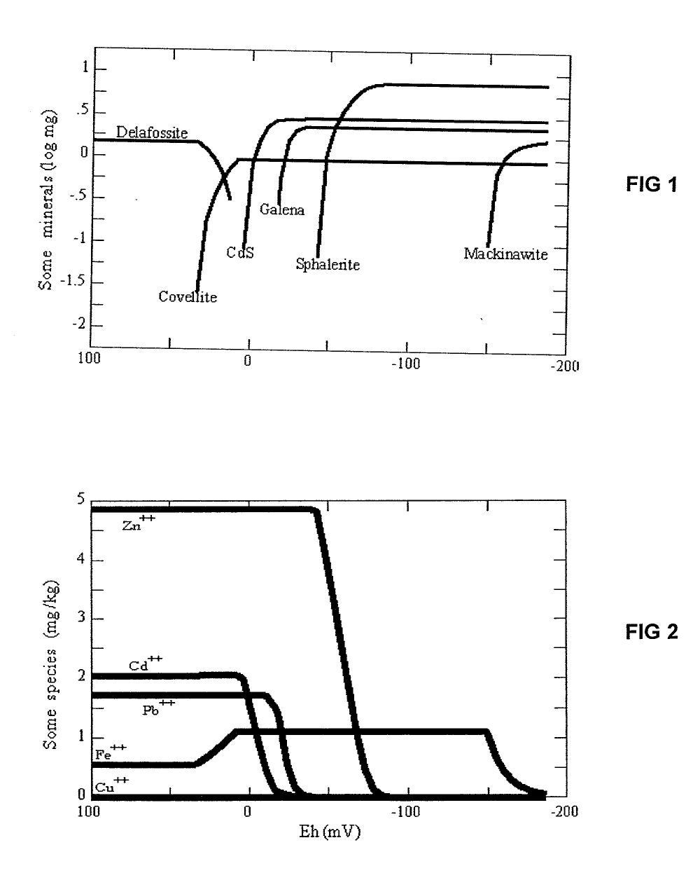

FIG. 1 illustrates part of the science behind the present invention. FIG. 1 illustrates a model in which an aqueous solution containing Cu, Cd, Pb, Zn, and Fe ions is subjected to increasingly reducing conditions (aqueous sulfide concentration increases left to right). As conditions become more reducing, oxide minerals like delafossite (CuFeO₂) dissolve and release metal ions into the solution. The level of sulfide ions in solution will slowly increase until the solubility of the first metal sulfide species is exceeded. During precipitation of this metal sulfide, the redox potential is buffered, since the precipitating metal sulfide removes sulfide from the solution. In this model system, the first formed sulfide is covellite (CuS). Covellite will precipitate until most of the Cu²⁺ ions are removed from solution. After the copper ions are depleted, aqueous sulfide again increases until saturation is reached with respect to the next metal sulfide, in this case greenockite (CdS). This process will continue as sulfide is produced until all of the available metals in turn are precipitated sequentially as the metal sulfide minerals galena (PbS), sphalerite (ZnS), and mackinawite (FeS). FIG. 2 illustrates the calculated metal ion concentrations in the system plotted against increasing concentration of sulfide. Note that the concentration of each metal ion decreases dramatically following each precipitation event. It is envisioned that this phenomenon can be implemented in a controlled system by manipulating the rate of sulfide production relative to the rate of supply of the metal ion. Minerals precipitate over narrow Eh ranges, and the solution composition can be manipulated to spatially separate Eh ranges where each specific mineral is formed, permitting the recovery of pure metal sulfides.

It is envisioned that this method can be implemented in a controlled system in which the rate of change in the concentration of sulfide ions is controlled. The rate of change in sulfide ion concentration is, in turn, the result of the growth of sulfide-reducing bacteria, and it is that growth that is controlled to achieve the desired slow rise in sulfide concentration. It is envisioned that a flow-through reactor has separate chambers that are controlled to have different and specific sulfide concentrations in each chamber. The rise in sulfide concentration can be manipulated such the minerals precipitate (and thus aqueous sulfide concentrations) in spatially separate chambers. In those spatially separate chambers, recovery of the precipitating particles will yield pure metal sulfide of the metal being precipitated in each chamber.

There are many species of sulfate-reducing bacteria, and a subset can be purchased. In the examples below, mixed cultures and commercially available cultures are used to demonstrate a proof of principle experiment, but other suitable strains can be readily isolated from the environment as well. All that is required is that the chosen bacterium, or mixed culture of sulfate reducing bacteria, grow in a controllable manner.

Most methods of metal recovery are energy expensive. The system and method described here is potentially inexpensive because it operates at (or below) room temperature. The method could be utilized to recover metals from acid mine drainage, bioleaching plants, or other commercial fluids or waste streams. The strategy has a clearly articulated and defined scientific basis. It has been shown to operate under certain natural conditions, indicating potential for this technology in in situ mine remediation. The approach has been shown to work in simple batch reactor systems using both mixed cultures and commercially available bacterial species. The technology is logically developed into a flow-through reactor in order to achieve relatively stable operating conditions consistent with selective and sequential extraction of metals as nanoparticulate metal sulfides.

### EXAMPLES

The natural system: “proof of concept” in the field.

Sulfate reducing bacteria (SRB) are nearly ubiquitous in low- to medium-temperature (5-40° C.) anoxic natural environments. In addition, some species are thermophiles or extreme thermophiles (and can grow at temperatures in excess of 100° C.). It is well known that dissolved metals react with aqueous sulfide produced by SRB, resulting in precipitation of metal sulfide minerals. However, the formation of distinct zones in which individual sulfides form as nearly pure single phases of single metals has only been recently recognized in modem environments. This phenomenon requires that the rate of supply of fluids transporting the metals into the system is fast compared to the rate of sulfide generation. In general, this state is achieved by limiting the flux of organics into the system (thus the rate of metabolism and generation of sulfide, as outlined above).

The Piquette mine site, near Tennyson, Wis., offers an excellent example of how bacterially-mediated separation of sulfides minerals works, even in a complex natural system. Within the pale-colored biofilms of SRB found in the mine, sulfide levels are buffered by reaction with dissolved zinc (0.09-1.1 ppm concentration). The result is formation of almost pure nanocrystalline (1.3-˜10 nm diameter) ZnS (sphalerite/wurtzite). The ZnS particles flocculate to form spheroidal aggregates (typically 100 nm-2 μm in diameter). In this case, Zn solutions are supplied by slow groundwater flow and organic compounds are released by slow degradation of mine timbers

Addition of organic substrates in proximity to the sample site for the sphalerite crystals resulted in the formation of mixed ZnS and iron sulfide mineral assemblages. This result is anticipated, given that the increased supply of organic compounds will stimulate the activity of SRB, overwhelming the capacity of the system to buffer the sulfide concentration by ZnS precipitation.

Laboratory Proof of Concept

Experiments were conducted with an enrichment culture grown from the ZnS-bearing biofilm from the Piquette mine described above. Microorganisms were cultured anaerobically at room temperature using medium DSMZ 63 (Table 1 below), which is formulated to select for growth of SRB. Once the cultures became visibly turbid, aliquots were sub-cultured into the experimental media for mineral precipitation experiments. The experimental media consisted of DSMZ 63 with variable amounts of ZnSO₄.7H₂O (0-92%) substituted in for the FeSO₄.7H₂O. The treatments were 8% Fe-92%Zn, 16% Fe-84% Zn, 32% Fe-68% Zn, and 100% Fe. Experiments were conducted in sealed 100 ml serum bottles with approximately 50 ml media. After the initial inoculation, a redox-sensitive indicator (resazurin) used in the solutions turned colorless, indicating a change to anaerobic conditions. Cultures were allowed to grow for several days, until the media was visibly turbid and precipitates formed. In the experimental controls in which 100% of the added transition metal was Fe²⁺ (0% Zn), the precipitates were black; in all other treatments with Zn in the media the precipitates were whitish. Several days after inoculation, aliquots of the media (solution, cells, and precipitates) were collected using sterile syringes, and samples were analyzed by scanning and transmission electron microscopy (SEM and TEM).

For SEM analysis, approximately 0.5 ml of solution was filtered using a 0.1 μm polycarbonate filter. The precipitates were washed twice with approximately 1 ml of deionized water to remove soluble salts ftom the media. The filters were then placed on carbon tape on an aluminum SEM stub and allowed to air dry. Samples were gold coated before SEM analyses to prevent charging by the electron beam. Filters with cells and precipitates were analyzed with a Leo 1530 Field Emission Scanning Electron Microscope at the Material Science Center, UW Madison. SEM operating conditions for all samples were SE detection, 3 kV accelerating voltage and 4 mm working distance. All samples had cells with a wide range in size (from <1 to several μm) and morphology (cocci, rods, spirillum), though the most abundant morphology (>90%) was short rods approximately 1×2 μm in size. The bulk of the precipitates from Zn-bearing solutions were spherical aggregates that were approximately 20-200 nm size. Though smaller in size, these spherical aggregates resemble the ZnS precipitates in the biofilm. Much smaller (few to few tens nm sized) aggregates were also associated with the cells and adhered to the filter. The morphology of the precipitates in the Fe-only solution was different than that of precipitates formed in Zn-containing treatments. The bulk of the mineral precipitates was very fine-grained (few to few 10's nm sized) and occasionally elongated.

For TEM analysis, approximately 1 ml of the media suspension was filtered and rinsed twice with DI water. The filter was placed in a 1.5 ml eppendorf tube with approximately 0.5 ml DI water. The tubes were vortexed and sonicated to remove cells and precipitates from the filter. Approximately 10 μL of this cell/precipitate suspension was placed on a formvar coated 200 mesh Cu TEM grid and allowed to air dry. Grids were carbon-coated before TEM analysis. TEM work was performed using a Philips CM200 TEM at the Material Science Center, UW-Madison, operating at 200 kV accelerating voltage. The chemistry of the precipitates was qualitatively determined by TEM-based Energy Dispersive Spectroscopy (EDS).

Spherical and irregularly shaped precipitates observed in SEM images of products of the Zn-bearing treatments were also readily apparent in TEM images, some in close association with cell surfaces. Selected area electron diffraction (SAED) analysis of this material showed only diffuse rings, indicating the material was finely crystalline or amorphous. TEM EDS analyses of numerous precipitates with variable size, morphology, and proximity to cells confirmed a primarily Zn and S composition (other minor constituents are probably derived from the media when the solution is dried for TEM examination).

TEM EDS results showed that the precipitates in the Fe-only experiment were fine grained aggregates with irregular morphology. Many were comprised of Fe and S, but some contained only Fe. Subsequent experiments have shown that the FeS formed in similar experiments rapidly oxidizes. Thus, it is almost certain that the Fe-only chemistry reflects air oxidation of sulfide to ferric oxyhydroxides and (soluble) sulfate during sample preparation.

TABLE 1
DSMZ 63 medium used in enrichment culture experiments

| | |
|---|---|
| Solution A: 980 ml DI | |
| K2HPO4 | 0.5 g |
| NH4Cl | 1.0 g |
| Na2SO4 | 1.0 g |
| CaCl2 · 2H20 | 0.1 g |
| MgSO4 · 7H20 | 2.0 g |
| DL-Na-lactate | 2.0 g |
| Resazurin | 1 mg |
| Solution B: 10 ml DI | |
| FeSO4 · 7H2O | 0.5 g |
| Solution C: 10 mL DI | |
| Na-thioglycolate | 0.1 g |
| Ascorbic acid | 0.1 g |

Experimental Metal-sulfide Separation

The sulfate-reducing bacteria (SRB) used for this experiment were Desulfovibrio desulfuricans (DSM 642) and Desulfosporosinus orientis (DSM 765), two species commercially available through DSMZ (Deutsche Sammlung von Mikroorganismen und Zellkulturen—German Collection of Microorganisms and Cell Cultures, www.dsmz.de). Both species are capable of using lactate as a carbon source, but D. orientis growth is slowed relative to that observed when using pyruvate. Cultures were grown on DSMZ 63 using lactate as the carbon source and three different combinations of metal-sulfates for electron acceptors (FeSO₄ only, FeSO₄+ZnSO₄, and FeSO₄+ZnSO₄+CuSO₄). The medium was modified by dilution to 20% of recommended “stock” concentrations of reagents, and by substituting non-sulfate compounds for both MgSO₄ and Na₂SO₄. NaHCO₃ was also added. These experiments utilized batch (rather than flow through) reactors. An inoculum of 100 μl of each species was pipetted into 50 ml of each type of medium in sterile serum bottles; each series of inoculations was performed in triplicate. All cultures were incubated at room temperature in an anaerobic chamber for up to 10 days.

Turbidity indicative of exponential growth of D. desulfuricans was observed in some serum bottles after 2-3 days. Desulfovibrio cultures that were grown on medium with FeSO₄ as the only electron acceptor showed a slight darkening after 3 days (4-5 days for Desulfosporosinus). This darkness increased over time. The cultures turned completely black at about 5-6 days (6-7 days for Desulfosporosinus). The visible black material was identified as fine-grained FeS. Desulfovibrio cultures grown with both Fe- and Zn-sulfates contained a fine-grained white precipitate after 2-3 days (4-5 days, Desulfosporosinus) and fine grained black particles after 4-5 days (5-6 days, Desulfosporosinus). No growth or precipitation was observed for either species of SRB grown with Fe-, Zn- and Cu-sulfates.

Aliquots of 100 μl of medium containing both cells (both species) and precipitates were sampled from each serum bottle after 1, 3, and 5 days of incubation. 50 ml of each of these samples were filtered through a Millipore 0.1 μm polycarbonate filter system and precipitates were washed twice with ultrapure water. The filters were then mounted on aluminum stubs and coated with 20 nm of gold to prevent charging in the field-emission scanning electron microscope.

FESEM investigation of samples taken from cultures of D. desulfuricans showed abundant umnineralized, curved Desulfovibrio cells and a second type of densely-mineralized cell, tentatively identified as the spore-forming non-sulfate reducing bacterium Clostridium. Samples taken from Desulfosporosinus cultures contained rod-shaped D. orientis cells and few precipitates. In the samples taken from FeSO₄-containing Desulfovibrio cultures, aggregated particles were observed and yielded distinct peaks for Fe and S when analyzed with EDS. In the samples taken from Fe- and Zn-sulfate-using Desulfovibrio cultures, aggregates of sub-micron-sized spherical particles were observed. For samples taken after 1 day, these particles yielded sharp EDS peaks for Zn and S only. The absence of a significant peak for Fe in the first samples taken from the Fe- and Zn-sulfate-using Desulfovibio culture indicates that phase separation was achieved by the controlled growth of Desulfovibrio cells. After 3 and 5 days, the amount of Fe observed qualitatively in spectra increased. This is expected in a batch reactor system due to the depletion of Zn (as predicted by the theoretical model). This event corresponded approximately with the time when cell growth entered the exponential phase.

The presence of Clostridium-like cells in the “pure” culture of Desulfovibrio possibly resulted from contamination by Clostridium spores from Tennyson natural mixed-cultures, which were handled within the same anaerobic chamber. Regardless of their origin, the presence of these heterotrophs would not strongly affect the sulfide-forming reaction. In fact, the possible competitive scavenging of organics and nutrients by other non-sulfate reducing heterotrophs would actually promote sulfide phase separation by slowing the growth rate of SRB.

The “proof of concept” batch reactors are not intended as a model for a commercially viable system, as the product formed varies with time due to changing conditions in the reactor. An appropriate model for commercial use should be based on a flow-through system. This could be deployed in the field, where slow growth of SRB is the norm. Alternatively, a laboratory or commercial system could be designed using principles determined through analysis of the natural environment described above.

Design of an Effective Laboratory-scale “Flow-through” System

It is proposed here that a modified laboratory reaction system can be designed that uses a “flow-through” reaction vessel. The reaction chamber contains organic material of some type. Our current experiments utilize a column that contains wood-pulp or rejected unbleached paper products, because of the low cost of these byproducts of the paper and timber industries. The column is inoculated with sulfate-reducing bacteria. Following cell growth and sulfide production, the column will become “poised” with respect to reducing potential and metal-sulfide reactivity. A solution of mixed metals can then be introduced into the column from below and allowed to exit at the top of the column. Depending upon the specific organic substrate utilized, additional biologically-needed ions (e.g., phosphate) are added to solution. As noted above, the metals will react with H₂S to form metal-sulfide precipitates. The first (and only) product within the column will be the less soluble metal-sulfide phase so long as the rates of fluid flow are coupled to the rate of sulfide production (the flow rates and column length can be changed to optimize metal recovery). The system can be maintained via monitoring of the outflow solution composition (if loss of metals other than the target metal is observed, flow rates can be increased and/or concentrations of growth promoting constituents in the solution decreased). Subsequent columns colonized by SRB and optimized for increasingly reducing (sulfide-rich) conditions will allow extraction of additional pure sulfide phases (in order of increasing solubility). In this way, “zones” of metal-sulfide precipitation will be formed and a bacterially-mediated “chromatographic” separation of phases achieved.

It is understood that the invention is not confined to the particular embodiments set forth herein as illustrative, but embraces all such modified forms thereof as come within the scope of the following claims.

What is claimed is:

1. A method for extracting and segregating metals from an aqueous solution containing mixed metal ions, the method comprising the steps of
(a) exposing the solution to a slowly increasing concentration of sulfide ions to selectively precipitate metal sulfides from the solution; and
(b) recovering the metal sulfides as they precipitate.

2. A method as claimed in claim 1 wherein the increasing sulfide concentration is the result of the growth of sulfate reducing bacteria.

3. A method as claimed in claim 1 wherein the metal sulfides precipitate as very small crystalline spheres.

4. A method as claimed in claim 1 wherein the metals ions in the solution include at least two metals selected from the group consisting of copper, cadmium, lead, zinc, and iron.

5. A method for extracting and segregating metals from an aqueous solution containing mixed metal ions, the method comprising the steps of
(a) culturing in the solution a strain of sulfate reducing bacteria to generate a slowly increasing concentration of sulfide ions to selectively precipitate metal sulfides from the solution; and
(b) recovering the metal sulfides as they precipitate.

6. A method as claimed in claim 5 wherein the metal sulfides precipitate as very small crystalline spheres.

7. A method as claimed in claim 5 wherein the metals ions in the solution include at least two metals selected from the group consisting of copper, cadmium, lead, zinc, and iron.

8. A reactor useful for the extraction and temporal segregation of metal ions from an aqueous solution of mixed metal ions, the reactor comprising
a vessel containing a solution of mixed metal ions; and
a culture of sulfate reducing bacteria which generate an increasing concentration of sulfide ions in the vessel to selectively precipitate out individual species of metal sulfides.

9. A reactor as claimed in claim 8 wherein the vessel is flow-through.

10. A reactor as claimed in claim 8 wherein the vessel is a batch reactor.

11. A reactor as claimed in claim 8 wherein the vessel includes an organic substrate on which the bacteria may grow.

12. A flow-through reactor useful for the extraction and spatial separation of metal ions from an aqueous solution of mixed metal ions, the reactor comprising
a vessel containing a solution of mixed metal ions,
an organic substrate on which sulfate-reducing bacteria may grow, and
a culture of sulfate reducing bacteria which generate an increasing concentration of sulfide ions in the vessel to sequentially precipitate out individual species of metal sulfides.
